(19) United States
(12) Patent Application Publication
(10) Pub. No.: US 2012/0289947 A1
(43) Pub. Date: Nov. 15, 2012

(54) DEVICE AND METHOD FOR REMOVING VEINS

(76) Inventor: Wolfgang Neuberger, Dubai (AE)

(21) Appl. No.: 13/522,885

(22) PCT Filed: Jan. 18, 2011
(86) PCT No.: PCT/US11/21496

§ 371 Date: Jul. 18, 2012

Related U.S. Application Data
(60) Provisional application No. 61/295,916, filed on Jan. 18, 2010.

(51) Int. Cl. A61B 18/22; A61B 18/20

(52) U.S. Cl. 606/3; 606/13; 606/15

(57) ABSTRACT

A device and method for safe, substantially blood-free vessel removal that preserves vein patency are disclosed. A specially designed lens-free laser vessel stripping device is inserted into the body and coupled to a laser energy source, to remove a major vessel while cutting and coagulating its lateral branches with minimal damage to surrounding tissue. Energy source is preferably a diode laser source emitting at wavelengths of about 980 nm, about 1470 nm or a combination of these wavelengths for obtaining best ablative and coagulative effects, thus achieving a safe and efficient removal of vessels with minimum collateral damage and thus faster recovery. Veins harvested using this device and method are better candidates for autologous grafts in other surgeries. Device and treatment are proposed for, but not limited to vein stripping of insufficient varicose veins, and removal of healthy veins for use as autologous grafts in surgical procedures such as coronary bypass surgery and for treatment of Peyronie's disease.

### RELATED CASE INFORMATION

This application claims the benefit of U.S. Provisional Application Ser. No. 61/295,916 by Wolfgang Neuberger, entitled “Improved Device and Method for Removing Veins” filed Jan. 18, 2010, which is incorporated by reference herein.

### BACKGROUND OF THE INVENTION

1. Field of the Invention

The present invention relates to laser vascular treatments and in particular, to the use of laser energy through one or more conveying means to remove veins for treatment of vascular pathologies and in connection with vein harvesting for other surgeries.

2. Information Disclosure Statement

The human venous system of the lower limbs consists essentially of the superficial venous system and the deep venous system, both connected by perforating veins. The superficial system comprises the great and the small saphenous veins, while the deep venous system includes the anterior and posterior tibial veins, which converge to form the popliteal vein near the knee. The popliteal vein, in turn, becomes the femoral vein when joined by the small saphenous vein.

The venous system comprises valves, whose main function is to achieve unidirectional blood flow back to the heart. Venous valves are usually bicuspid valves, with each cusp forming a blood reservoir, which force their free surfaces together under retrograde blood pressure. As a consequence, when properly operating, retrograde blood flow is prevented, allowing only antegrade flow to the heart. A valve becomes incompetent when their cusps are unable to seal properly under retrograde pressure gradient, so retrograde blood flow occurs. When retrograde blood flow occurs, pressure increases in the lower venous sections, dilating veins and usually leading to additional valvular failure.

Valvular failure, usually referred to as venous insufficiency, is a chronic disease that can lead to skin discoloration, varicose veins, pain, swelling and ulcerations. Varicose veins refer to blood vessels that have become enlarged and twisted and have progressively lost their wall elasticity. Owing to the widening of the blood vessels, vein valves cannot be completely closed and veins lose their ability to carry blood back to the heart. This leads to an accumulation of blood inside the vessels, enlarging and twisting the veins even more. Furthermore, varicose veins usually have a blue or purple color and may protrude twisted above the surface of the skin, being responsible of their characteristically unattractive appearance. They are commonly formed in the superficial veins of the legs, which are subject to high pressure when standing. Other types of varicose veins include venous lakes, reticular veins and telangiectasias.

There is a number of treatments available intending to eradicate these kinds of vascular pathologies. Some of them only consist in relief of symptoms because they do not prevent new varicose veins from forming. These include elevating the legs by lying down or using a footstool when sitting, elastic stockings and exercise.

Varicose veins are frequently treated by eliminating the insufficient veins. This forces the blood to flow through the remaining healthy veins. Various methods can be used to eliminate the problem insufficient veins, including, surgery, sclerotherapy, electrocautery, and laser treatments.

Surgical removal of insufficient veins is done by a technique called vein stripping. The goals of vein stripping are to relieve pain, to improve circulation through the venous system by removing pathways of blood reflux and to improve the esthetic appearance. For insufficient saphenous vein, stripping involves its removal from the leg and any varicose tributary veins. The classical method of saphenous vein stripping is the Babcock method which comprises the following steps: once the vein is identified, physician ties off the main tributary veins associated with it and interrupts blood flow between the saphenous vein and the femoral vein. A wire is introduced into the free end of the saphenous vein and advanced down its length and out through a second incision made usually at the upper calf, just below the knee. The end of the vein nearest the groin is tied making a tight knot, and then the knot is tied to the end of the wire. Smaller veins connected to the saphenous vein may be cut away with tiny incisions. Physician then pulls the wire downward and as the wire advances through the length of the vein it carries the vein along with it, turning the vein inside out and pulling it away from the smaller tributary veins. Usually a considerable force must be made to achieve this. The vein is removed through the incision in the upper calf. In some cases vein is stripped all the way to the ankle. Finally, incisions are closed with stitches and compression bandages should be used. Despite its effectiveness, this method presents numerous disadvantages. Many of the side branches of target vein are pulled out together with major vein. This causes considerable bleeding and must be controlled with pressure bandages. Also, a considerable amount of tissue attached to the major vein and side branches are pulled out, thus causing severe trauma to peripheral tissue. This causes considerable pain and therefore requires administration of analgesic drugs. Furthermore, patient must usually remain hospitalized for a few days and will need to wear the pressure bandages for several weeks after the procedure.

In an attempt to overcome mentioned disadvantages, Redtenbacher et al. discloses in U.S. Pat. No. 6,858,027 a vein stripper for removing diseased veins, comprising a probe that is inserted into the vein; and a coagulation and cutting instrument having an electrode assembly which includes at least two electrodes arranged at one end of the coagulation and cutting instrument. Radiofrequency is used for coagulating and cutting. This device for performing vein stripping has important limitations due to hazards involved with radiofrequency. For example, nerve tissue, e.g. saphenous nerve in the lower part of the leg, can be damaged when working with this instrument. One drawback of RF methods is that they require maintained contact between the RF electrodes and the vein wall and thus deliver energy to the vein wall essentially only through such points of contact. Yet another drawback of RF methods is that they can be more time consuming and thus more stressful to the patient than otherwise desired. In addition, RF catheters and electrodes can be relatively complex and more expensive to manufacture than otherwise desired. Furthermore patients wearing pacemakers or other electronic implantable devices are in potential danger due to risk of interference of such devices due to radiofrequency waves and therefore they are not recommendable candidates for use of this device.

In U.S. Patent Publication No. 2009/0018563, Redtenbacher et al. describe a vein stripping device for removal of varicose veins including a base body and a guide cable connected to it and insertable into a varicose vein. A lens is attached to the base body and receives radiation from a Holmium-YAG laser source or a CO₂ laser source at a wavelength of 2 μm or longer. This patent presents numerous disadvantages. It is well known by those skilled in the art that different light sources at different wavelengths will cause altogether different effects on target tissue. Wavelength range claimed may not be ideal for achieving desired combination of cutting and coagulation effects, two important factors which determine the outcome success of the procedure. For instance, wavelengths of 2 μm or more, emitted by Holmium-YAG laser sources, are known as having poor penetration properties and therefore are not ideal for achieving good coagulation effect, which is highly desired to prevent bleeding in this type of procedure. In addition, pulsed holmium lasers usually emit radiation in narrow pulses. Thus, in order to achieve appropriate energy levels for producing certain effects on tissue, laser power should be high. High power radiation applied in short bursts generally creates undesired shockwaves, which in turn produce undesirable and unpredictable effects on tissue. As a consequence, holmium lasers may not be recommendable for applications in which precise amounts of energy are to be applied and non-linear processes must be avoided. Another disadvantage is that this system requires lens arrays to focus laser beams in order to achieve desired results. Laser devices including lens arrays usually present a complex and costly design. Additionally, lenses are subject and sensible to getting dirty, misty, become unattached or get even damaged when used within human tissue and this may affect proper performance of the whole laser system. Moreover, after some time of use, lens transparency is usually impaired, thus dissipating a considerable amount of laser energy as heat, resulting in laser energy emitted by the source not being transferred completely to tissue, compromising precision and selection of treatment parameters. Finally, size and cost are important issues to take into account. For example, diode lasers have numerous advantages over ionic crystal lasers and gas lasers. Among them, higher output, at reduced dimensions and weight. They also have simpler and smaller air cooling systems. Moreover, being integrated with optical fibers, they have a high reliability and do not need alignment.

As can be seen, a safe and effective method of stripping insufficient veins remains elusive.

There are situations in which medical treatment involves removal of healthy vessels. This is commonly known as harvesting. For instance, harvesting of the saphenous vein tract has been proposed in the treatment of coronary artery disease (CAD) and treatment of Peyronie's disease.

CAD occurs when atherosclerotic plaque (hardening of the arteries) builds up in the wall of the arteries that supply the heart. This plaque is primarily made of cholesterol. Plaque accumulation can be accelerated by smoking, stress, high blood pressure, elevated cholesterol, and diabetes. Patients are also at higher risk for plaque development if they are elderly, or if they have a positive family history for early heart artery disease.

The atherosclerotic process causes significant narrowing in one or more coronary arteries. When coronary arteries narrow more than 50 to 70%, the blood supply beyond the plaque becomes insufficient to meet the increased oxygen demand during exercise. The heart muscle in the territory of these arteries becomes ischernic. Patients often experience chest pain (angina) when the blood oxygen supply cannot keep up with demand. When a blood clot (thrombus) forms on top of this plaque, the artery becomes completely blocked causing a heart attack.

Coronary artery bypass surgery (CABG) is a surgical procedure performed to relieve angina and reduce the risk of death from CAD. Vessels elsewhere in the patient's body are grafted to the coronary arteries to bypass atherosclerotic narrowed passages.

CABG surgery has been shown to improve long-term survival in patients with significant narrowing of the left main coronary artery, and in patients with significant narrowing of multiple arteries, especially in those with decreased heart muscle pump function.

The most commonly used vessel for the bypass is the saphenous vein from the leg. Saphenous vein is harvested and graft is sown to the coronary arteries before the narrowing or blockage. The other end of this vein is attached to the aorta.

Greater saphenous vein harvesting is now a routine procedure, but long-term patency of the graft is linked to its harvesting technique. The vein must be handled with great care in order to minimize trauma to its intima and adventitia. Also, collaterals must be carefully ligated or clipped in order to avoid tearing or secondary bleeding.

Typical state of the art procedures for harvesting a saphenous vein are tedious, time consuming, and cause considerable trauma.

One such procedure is open surgery. An incision is made along the leg for a length corresponding to the length of the graft required, and vein is transected and stripped from the leg. In detail, the method consists in identifying the saphenous vein distally, making an incision adjacent to the vein, dissecting to visually identify the vein and finally beginning to expose the vein by surgical incision to the entire desired length. The vein is then sharply dissected and freed completely from the surrounding tissue. Side branches are tied on the vein side and surgical clips are placed adjacently. Next, the branch is divided. Once the vein is free, surgical clips and ties are placed on the distal and proximal ends and these areas are transected. A vein catheter is introduced into the distal end of the saphenous vein, tied in place and heparinized saline is injected to identify any leaks that need repair. The vein is repaired with sutures and is then flushed with cold heparinized blood and placed in a small cup of the same solution until ready for use. This surgery is traumatic, has high recovery time and hospital stay and adds cost to the procedure.

Another procedure is by use of an endoscope. In this method, a few small incisions are made on the leg over the saphenous vein. The saphenous vein is transected and ligated at its ends and the endoscope is inserted into the small incisions. While visualizing the vein with the endoscope, the entire length of the vein is harvested by slow dissection. The endoscope is advanced under the skin along the saphenous vein's length while transecting and ligating its connecting branches until the entire segment of the saphenous vein is free and able to be removed. Benefits of this method include lower wound morbidity, faster recovery, and better patient satisfaction in comparison to the open technique. This method requires only a few small incisions and is therefore less traumatic. Apart from its obvious improved esthetic results, this technique markedly reduces local pain. However, it is a complex procedure and requires a substantial amount of time. Endoscopic methods have not gained widespread acceptance among vascular surgery specialists because many consider the endoscopic systems somewhat cumbersome. Moreover, this method requires additional instrumentation and setup. Additionally, endoscopic harvesting of veins for coronary artery bypass graft surgery may compromise long-term outcomes, as researchers found. Compared with open surgery to harvest grafts, endoscopic procedures had higher 12- to 18-month vein-graft failure. Finally, endoscopic harvesting is associated with significantly higher mortality and myocardial infarction rates.

An inversion method has been described for harvesting vein grafts that does not require endoscopy or fluoroscopic visualization. By using a coaxial guide wire and catheter system, the great saphenous vein is completely removed inside out from its bed. The inversion method provides opportunity for the surgeon to excise valve leaflets under direct vision. The vein can then be turned “right side in” again for use as an arterial bypass graft. Tributaries that are large enough to potentially damage the vein generate force feedback that tells the operator to stop pulling the inversion catheter. Traction on the tributary causes the skin to dimple, which marks the site for a short skin incision through which the operator palpates the guide wire with a fingertip and identifies the tributary to be divided. This method is in essence very similar to the conventional vein stripping method and therefore presents similar disadvantages.

Peyronie's disease is a connective tissue disorder involving the growth of fibrous plaques in the soft tissue of the penis affecting as many as 1-4% of men. Specifically the fibrosing process occurs in the tunica albuginea, a fibrous envelope surrounding the penile corpora cavernosa causing an abnormal curvature of the penis. Peyronie's disease causes a bent penis during erection. A hard, fibrous layer of scar tissue develops under the skin on the upper or lower side of the penis. When the penis is erect, the scar tissue pulls the affected area off at an angle, causing a curved penis. The condition can cause pain and make sexual intercourse difficult. It can be a physically and psychologically devastating disease. Without treatment, about 12-13% of patients will spontaneously improve over time, 40-50% will get worse and the rest will be relatively stable.

Peyronie's disease treatments involve nonsurgical and surgical approaches. If Peyronie's disease doesn't improve on its own, oral medication such as Colchicine, Potassium aminobenzoa, and Vitamin E is indicated. This treatment appears to be effective only for the first 12 to 18 months, while plaque is still forming. Intralesional injections such as collagenase, calcium channel blockers or interferons directly into the plaque may be administrated. These drugs are intended to break down scar tissue deposits and return the tissue-building process to normal. Multiple injections are applied over a period of months. The success of intralesional injections varies. When these methods of treatment are not effective, surgery is an option. Surgery is generally effective at restoring normal erections. One common surgical method is the Nesbit application. Tissue on the unaffected side of the penis is shortened, canceling the bending effect. However, this type of surgery can shorten the penis. A preferred method is plaque incision with saphenous vein graft. Several linear cuts are made in the plaque, which allows straightening. The cut plaque is then covered with a grafted vein. This procedure is generally used in men who have a shorter penis, a curve of more than 45 degrees or an hourglass-shaped deformity.

Harvesting of the saphenous vein tract by means of leg stripping is proposed in the treatment of Peyronie's disease. For example, one technique describes a W-shaped saphenous vein after plaque incision to correct severe penile deformity associated with Peyronie's disease. Graft material is obtained from the lower saphenous vein by means of distal ‘leg short stripping’ technique. Once again, conventional stripping methods, cause considerable bleeding and must be controlled with pressure bandages. Also, a considerable amount of tissue attached to the major vein and side branches are pulled out, thus causing severe trauma to peripheral tissue causing considerable pain and therefore requiring administration of analgesic drugs.

As can be seen, a safe and effective method of harvesting healthy veins remains elusive.

There is thus a need for a safer and more effective method of stripping insufficient veins. There is also a need for a simpler and less invasive method of safely and effectively harvesting vessels for use as autologous graft. Present invention addresses these needs.

### OBJECTIVES AND BRIEF SUMMARY OF THE INVENTION

It is an objective of the present invention to provide a device and method for improved treatment of vascular disorders.

It is also an objective of the present invention to provide a device and method for safer and more reliable removal of insufficient veins.

It is another objective of the present invention to carry out accurate safe removal of vessels, by using a localized energy source and conveying means.

It is still another objective of the present invention to perform vein stripping of dysfunctional vessels minimizing bleeding and trauma and thus diminishing hospital stay.

It is yet another objective of the present invention to perform accurate, safe removal of vessels for use as autologous prosthesis in graft placement procedures.

Briefly stated, a device and method for safe, substantially blood-free vessel removal that preserves vein patency are disclosed. A specially designed lens-free laser vessel stripping device is inserted into the body and coupled to a laser energy source for removing a major vessel while cutting and coagulating its lateral branches with minimal damage to surrounding tissue. Energy source is preferably a diode laser source emitting at wavelengths of about 980 nm, about 1470 nm or a combination of these wavelengths for obtaining best ablative and coagulative effects, thus achieving a safe and efficient removal of vessels with minimum collateral damage and thus faster recovery. Veins, harvested using this device and method, are better candidates for autologous grafts in other surgeries. Device and treatment are proposed for, but not limited to vein stripping of insufficient varicose veins and removal of healthy veins for use as autologous grafts in surgical procedures such as coronary bypass surgery and for treatment of Peyronie's disease.

### BRIEF DESCRIPTION OF FIGURES

FIG. 1a is a schematic representation of preferred embodiment of vein removal device.

FIGS. 1b and 1c are preferred configurations of the present invention including guide wire.

FIG. 3 depicts a flow diagram showing main steps of disclosed method using embodiment configuration described in FIG. 1b.

FIG. 4 depicts a flow diagram showing main steps of disclosed method using embodiment configuration described in FIG. 1c.

### DETAILED DESCRIPTION OF PREFERRED EMBODIMENTS

Numerous approaches have been proposed for removing insufficient veins. These present, as mentioned previously, several disadvantages including drawbacks related to patient discomfort, length and complexity of treatment, and inappropriate technology in the case of some RF and laser device approaches.

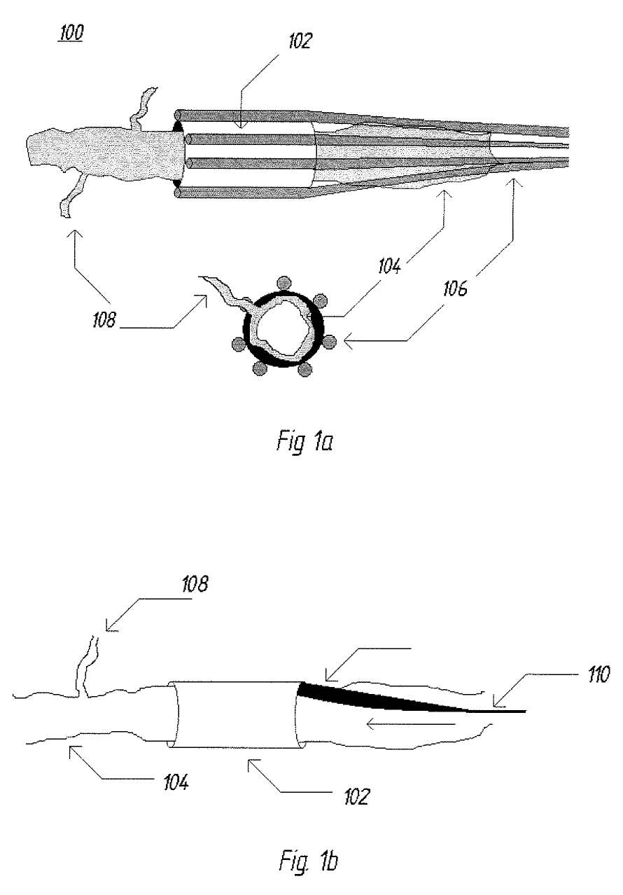

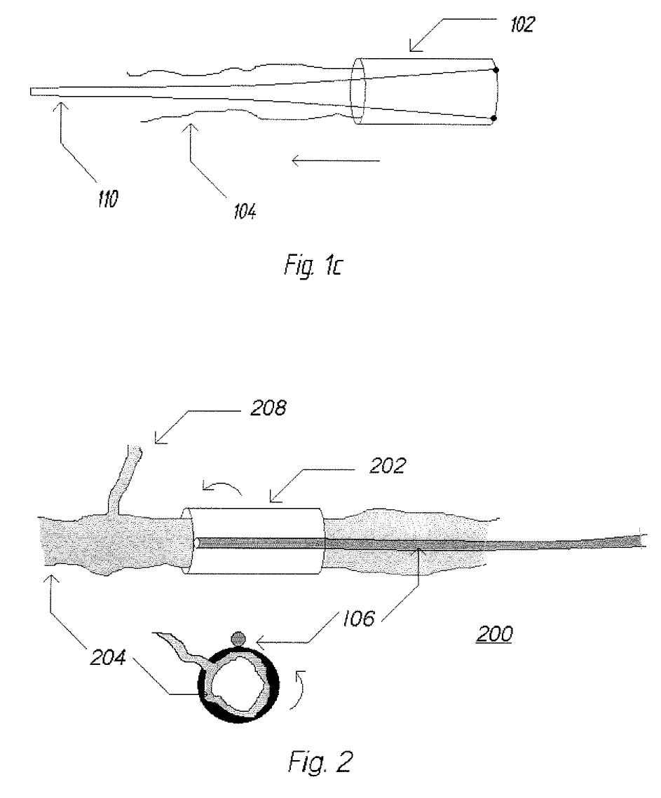

The present invention discloses method and device designed to perform safe, accurate and efficient vessel removal combined with lateral branches (tributaries) disconnection. While disconnecting tributaries, coagulation is carried out to prevent their bleeding. Removal of vein is rendered simple and safe, with minimum bleeding and without unnecessarily removing neighboring tissue. In a preferred embodiment, this is achieved with a device such as the one shown in FIG. 1a. Stripping device 100 is comprised of a cylindrical device 102 with an inner diameter that is slightly larger than diameter of vein 104 to be removed. A variable number of optical fibers 106 are attached around cylindrical device 102. Cylindrical device 102 is advanced along outside of vein 104. As device 100 is advanced, laser energy is applied, thus separating perivenous tissue from vein 104. Also, when a tributary vein 108 is encountered, laser energy conveyed from optical fibers 106 separate it from main vein 104 and coagulates blood leakage resulting from detachment. Each optical fiber may convey light energy from one or more diode laser source emitting at a wavelength or a combination of wavelengths adequate for achieving tissue ablation and coagulation. In another embodiment, some optical fibers emit at wavelengths that are preferably ablative and other optical fiber emit at highly coagulative wavelengths. Optical fibers emitting at different wavelengths are arranged in an intercalated manner.

In another preferred embodiment, as shown in FIG. 1b, cylindrical device 102 is placed around vein 104 to be removed as explained previously and guide wire 110 is attached at its back end. Guide wire 110 is then pushed forward together with cylindrical device 102 along entire length of vein 104 to be removed as laser energy is emitted simultaneously. Once device's trajectory is complete, vein 104 is free from tributaries and other neighboring tissue and can be easily removed either manually or with the help of a proper catheter. Vein is thus removed with minimal damage and is therefore suitable for use as autologous graft in different prosthesis applications.

In yet another preferred embodiment, as shown in FIG. 1e, guidewire 110 is inserted endovascularly until front end sticks out of vein 104 and then back end is attached to cylindrical device 102. As laser energy is applied, guide wire 110 is pulled, carrying cylindrical device 102 along with it. The action of pulling cylindrical device 102 back, hauls along with it vein 104 as it is being detached from neighboring tissue and tributaries. Surgeon feels resistance when a tributary 108 is reached, in which case he should diminish moving speed to make sure tributary is completely separated and no bleeding occurs. This embodiment allows for vein to be completely removed free of tributaries and minimum surrounding tissue in a single and simple pullback procedure.

FIG. 2 shows another embodiment of the present invention where stripping device 200 is comprised of a cylindrical device 202 with an inner diameter that is slightly larger than diameter of vein 204 to be removed. On outer surface of cylindrical device 202, a single optical fiber 206 is attached. Cylindrical device 202 is coupled to a spinning system which can rotate together with fiber in one or both directions or in one complete 360 degree turn in both directions, alternatively. As laser energy is emitted, cylindrical device 202 is advanced along outside of vein and simultaneously rotated. Rotational speed is set accordingly, considering parameters such as longitudinal speed, lasing parameters and nearby presence of vein tributary. Including a single optical fiber 206 renders device design simpler and rotational movements in combination with laser emission enhance tissue separation and tributary 208 detachment.

In a preferred embodiment laser source is a diode laser source emitting a wavelength of 1470±60 nm, 980±30 nm or a combination of both wavelengths in preselected proportions.

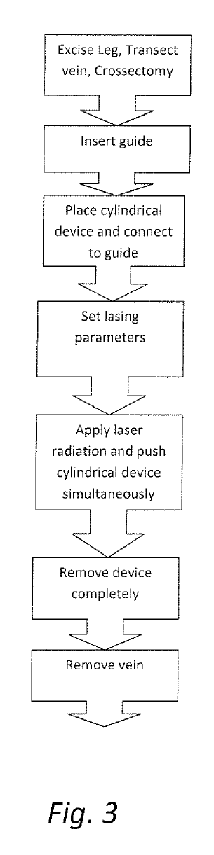

In a preferred embodiment, treatment method used to achieve effective vein harvesting is disclosed. FIG. 3 shows a flow diagram of successive necessary steps for a successful laser vein removal procedure using disclosed device. Procedure for saphenous vein can be summarized as follows: the leg is incised, at the upper and lower position between which the vein is to be removed and the vein is located in both positions. At both ends, vein is transected and a crossectomy is performed at proximal end and this end of the vein is ligated. Cylindrical device is placed outside of vein as explained in embodiment depicted in FIG. 1b and guide cable is attached to back part of cylindrical device. Now laser source is turned on and energy is applied while guide cable is gently pushed forward from the proximal position. As cylindrical device is pushed along the vein, and simultaneously energy is applied throughout the procedure, perivenous tissue is separated from vein and side branch ends will be cut and coagulated (cauterized). Thus, the peripheral end of the detached tributary is immediately ligated by coagulation, and therefore no further bleeding from this venous side branch should occur. Surgeon may feel resistance when a tributary is reached, in which case he should diminish pushing speed to make sure tributary is completely separated and no bleeding occurs. As surgeon keeps pushing on the guide cable, the cylindrical device will become eventually visible at proximal end of the stripping channel. When this happens, the surgeon stops application of energy and removes device from the leg by pushing it completely out. Then surgeon removes loosened vein manually or with the help of an appropriate catheter. If procedure was done for harvesting vein to use as autologous graft, measures to preserve removed vessel must be taken. Finally, surgeon proceeds to carry out standard close-up procedures. If procedure was carried out as treatment of insufficient varicose vein, because it is minimally invasive and bleeding and harm to surrounding tissue is kept at a minimum, no pressure bandages would need to be applied and thus, patient is shortly dismissed.

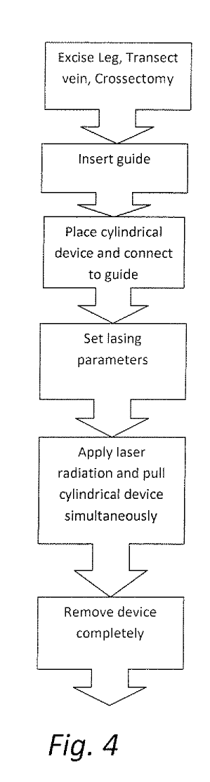

In another preferred embodiment, a treatment method used to achieve effective vein removal of veins is disclosed. FIG. 4 shows a flow diagram of successive necessary steps for a successful laser vein removal procedure using disclosed device. Procedure can be summarized in a similar case as was done in method described by FIG. 3. In this case, energy is applied while guide cable is gently withdrawn from the proximal position. As cylindrical device is pulled, it will pull vein out along with it. Simultaneously, as energy is applied throughout the procedure, side branch ends will be cut and coagulated (cauterized). The action of pulling cylindrical device back, hauls along with it vein as it is being detached from neighboring tissue and tributaries. This embodiment allows for vein to be completely removed free of tributaries and minimum surrounding tissue in a single and simple pullback procedure. Here again, because procedure is minimally invasive and, bleeding and harm to surrounding tissue is kept at a minimum, patients treated for vein insufficiency would not need pressure bandages and are thus dismissed shortly.

Disclosed device and method allow for an effective, safe removal of diseased and healthy vessels. This represents an essential procedure destined to but not limited to vein stripping of insufficient varicose veins and to removing of healthy veins such as the saphenous vein for use as autologous grafts in surgical procedures such as coronary bypass surgery and treatment of Peyronie's disease.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A lens-free laser medical device/system for use with vein stripping procedures to achieve safe, substantially blood free, removal of veins to either correct for venous insufficiency or to provide veins as autologous grafts in surgical procedures.

2. The lens-free laser medical device/system according to claim 1, wherein a diode laser source is used to complement vein stripping/removal instruments/techniques.

3. A lens-free laser medical device/system for improved removal off diseased or healthy veins, wherein a diode laser operating at a wavelength selected from the group consisting of 980+30 nm, 1470+60 nm and a combination of these wavelengths.

4. The lens-free laser medical device/system according to claim 3, comprising a diode laser source, at least one optical fiber to transmit light energy from a laser source, and vein stripping apparatuses.

5. A method for safe, substantially blood free removal of veins for either correction of venous insufficiency or for autologous grafts in subsequent surgeries using vein stripping and a laser source to ablate and coagulate treatment sites as required.

6. The method for removal of veins according to claim 5, comprising the steps of:
a. marking off treatment site for length of vein to be removed;
b. making incisions at or near edges of said treatment site;
c. inserting tools for vein stripping, and placing distal end as required;
d. introducing optical fiber to treatment site;
e. irradiating while removing vein to coagulate blood and/or ablate perforating veins;
f. removing vein and closing off both ends, if to be used for autologous grafts; and
g. suturing incisions as necessary.
